(19) United States
(12) Patent Application Publication
(10) Pub. No.: US 2020/0046261 A1
(43) Pub. Date: Feb. 13, 2020

(54) SYSTEM FOR CORRECTING SHOULDER ALIGNMENT, ASSEMBLY OF A SYSTEM AND A FURTHER PROCESSING DEVICE, AND A COMPUTER PROGRAM PRODUCT

(71) Applicants: Richard A.J. SHEARER, London (GB); Arif MOVSUMZADE, Baku (AZ)

(72) Inventors: Ulviyya RUSTAMOVA, Ganja (AZ); Arif MOVSUMZADE, Baku (AZ); Fidan RUSTAMOVA, Ganja (AZ)

(73) Assignees: Richard A.J. SHEARER, London (GB); Arif MOVSUMZADE, Baku (AZ)

(21) Appl. No.: 16/485,804

(22) PCT Filed: Feb. 13, 2018
(86) PCT No.: PCT/EP2018/053578

§ 371 Date: Aug. 14, 2019

(30) Foreign Application Priority Data
Feb. 14, 2017 (NL) 2018371

(51) Int. Cl. A61B 5/11; A61B 5/00

(52) U.S. Cl. CPC A61B 5/1116; A61B 5/4576; A61B 2562/0219; A61B 2560/0238; A61B 2562/04; A61B 5/7405

(57) ABSTRACT

The present document relates to a system, and assembly and a computer program product. The system is suitable for correcting shoulder alignment, and comprises two sensor devices configured to be placed on a user's body, a controller receiving sensor signals from the sensor devices, and an alarm generator for generating alarm signals regarding a detected misalignment. The sensor devices comprise a first and a second sensor to be placed on a respective first and second upper thoracic part of the user's body. The first and second upper thoracic parts are respectively located on different lateral sides of the user's spine. The controller calculates an alignment status of the shoulders of the user from at least the first sensor signal and the second sensor signal, and operates the alarm generator for generating an alarm signal when the controller identifies the alignment status to be indicative of a misalignment of the shoulders of the user.

### BACKGROUND

Many hours of a person's lifetime, for large amounts of people, are nowadays often spent sitting behind desks or behind computer screens. The rapid worldwide distribution of smartphones and tablets has increased this problem significantly. It is well known that this time is often spent while assuming an incorrect posture, without the person realizing this while being concentrated on other tasks.

Various systems have been developed over time to monitor posture, such as to signify an incorrect posture to a user in time or to stimulate or even force the user to assume a different posture for his own good. Such systems typically rely on cloth attachable sensors. However, a disadvantage is that many sensors are typically required to provide an accurate detection. This in turn causes such systems to be unpopular, due to the amount of effort required to put them on and/or due to the inevitable visibility thereof to other persons. For these reasons, other systems have been developed that rely on one sensor, which may be designed to be ecstatically acceptable. Although such sensors overcome the above disadvantage of being less popular due to their visibility, the other disadvantage of reduced accuracy remains.

In addition to these disadvantages, a further disadvantage is that most systems primarily focus on correction of the spinal posture, in particular the straightening of the posture to ensure that users assume a straight-up posture during activities. Indeed, many body structural disorders such as back pains may be prevented by a correct straight-up posture. However, an additional causes of head, neck, back pains are thereby often overlooked.

### SUMMARY OF THE INVENTION

In view of the above, it is an object of the present invention to provide system that overcomes the abovementioned disadvantages.

To this end, there is provided herewith a system in accordance with claim 1. The invention provides a system for correcting shoulder alignment, the system comprising at least two sensor devices configured to be placed on a body of a user, a controller communicatively connected to the sensor devices such as to receive respective sensor signals therefrom, and an alarm generator configured to generate alarm signals to inform the user of a detected misalignment, wherein the at least two sensor devices comprise a first sensor to be placed on a first upper thoracic part of the body of the user and a second sensor to be placed on a second upper thoracic part of the body of the user, wherein the first upper thoracic part and the second upper thoracic part are respectively located on different lateral sides of a spine of the user, and wherein the first sensor provides a first sensor signal and the second sensor provides a second sensor signal; wherein the controller is configured for calculating from at least the first sensor signal and the second sensor signal, an alignment status of the shoulders of the user, and wherein the controller is further configured for operating the alarm generator for generating an alarm signal when the controller identifies the alignment status to be indicative of a misalignment of the shoulders of the user.

The system in accordance with the present invention consists of at least two sensors, a first and a second sensor, that are placed on either side of the spine of the user on the upper thoracic body part. Preferably, these sensors are placed on the back or on top of the shoulders. The both sensors provide sensor signals to a controller which analyzes these and is able to detect a misalignment of the shoulders of the user.

An correct posture of the shoulders, such as a misalignment thereof, may put the muscles and vertebrae in the neck and spine under too much strain. This, after some time, may result in various physical problems such as chronic pain and headaches. When doing desk work or computer work, but also while using handheld devices such as smartphones and tablets, user are concentrated on the screen which may often not reside in an optimal position with respect to the head. Moreover, incorrect setting of the user's desk chair or the use of a comfortable chair encourages slouching and bad posture of the shoulders. The present invention enables to determine a shoulder alignment status and from this detect an incorrect shoulder alignment. Moreover, the invention is based on the insight that by accurately determining whether the shoulders of the user are aligned, it can be established that the posture of the user is incorrect. By signaling an alarm to the user to stimulate him or her to realign the shoulders, the users posture can be effectively corrected. Therefore, it is not necessary to obtain information from a plurality of sensors distributed across the users body to obtain an accurate determination of the users posture and to effectively allow correction thereof. The alignment of the shoulders may simply be determined by using two sensors placed on either side of the user's spine and the upper body part region. The reference values are obtained by performing a calibration process, from which an incorrect alignment thereafter may easily be detected. Thereto, the system further comprises a memory for storing data, wherein the controller is further configured for, in response to receiving a calibration trigger performing a calibration process: obtaining the first sensor signal and obtaining the second sensor signal; calculating a reference alignment status from the first and the second sensor signal; and storing the reference alignment status in the memory.

In accordance with a second aspect there is provided an assembly of a system in accordance with the present invention and a further processing device, such as a mobile terminal or a handheld device, the further processing device being configured for receiving an alarm signal transmitted by the communication device included in the alarm generator and signaling the user, via an output device included on the further processing device, of a detected misalignment of the shoulders.

In accordance with yet a further aspect, there is provided a computer program product for use in a further processing device, the computer program product residing on a computer readable medium, such as a network attached server or data store, the computer program product comprising instructions for causing the further processing device to receiving an alarm signal transmitted by a communication device included in an alarm generator of a system for correcting shoulder alignment and signaling the user, via an output device included on the further processing device, of a detected misalignment of the shoulders.

### BRIEF DESCRIPTION OF THE DRAWINGS

The invention will further be elucidated by description of some specific embodiments thereof, making reference to the attached drawings. The detailed description provides examples of possible implementations of the invention, but is not to be regarded as describing the only embodiments falling under the scope. The scope of the invention is defined in the claims, and the description is to be regarded as illustrative without being restrictive on the invention.

In the drawings:

FIG. 1 schematically illustrates two examples of incorrect shoulder alignment;

FIG. 2 schematically illustrates examples of correct and incorrect postures;

FIGS. 3A and 3B schematically illustrate a system in accordance with a first embodiment of the present invention;

FIG. 4 schematically illustrates a system in accordance with a second embodiment of the present invention;

FIG. 5 schematically illustrates a system in accordance with the present invention;

FIG. 6 schematically illustrates a system mounted on a semi-rigid structure in accordance with an embodiment of the invention.

### DETAILED DESCRIPTION

The present invention relates to a wearable device and a mobile application that enables to detect misalignment of the shoulders of a user. As an additional feature, the system further allows to detect other deviations from a correct posture, and helps correct such deviations. The device comprises of at least two sensors, a microprocessor, one or more batteries, one or more vibrating actuators, and an alarm generator optionally including a Bluetooth module for communication with a further processing device, such as a hand held device or mobile phone (e.g. smartphone).

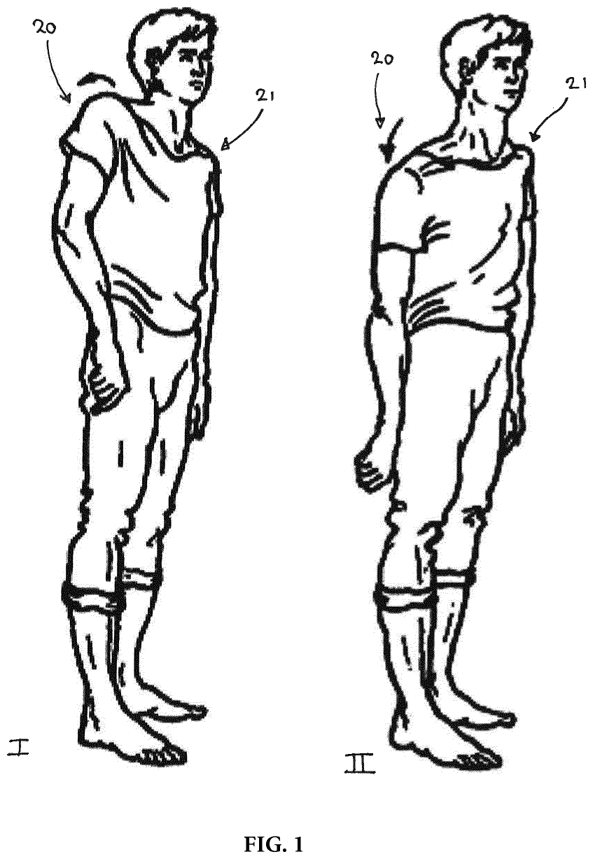

FIG. 1 schematically illustrates two examples of an incorrect shoulder alignment that may be detected by a system in accordance with the present invention. In the example on the left side of FIG. 1, the person illustrates has elevated his right shoulder 20 in an unnatural high position, which immediately triggers an alarm signal from a system in accordance with the present invention. In situation II on the right side of FIG. 1, the person is slouching and has his right shoulder 20 in a lower position than his left shoulder 21. This, likewise, is detected by a system in accordance with the present invention. As may be appreciated, a person sitting behind his desk behind a computer screen, concentrated on reading from the screen, may well assume positions such as illustrated in FIG. 1, situations I and II, while seated. For example, the situation illustrated in FIG. 1, I could be well assumed for a certain duration of time in case a person slouches in his seat while supporting his right elbow on an arm support. As may be imagined, also the position of FIG. 1, II may be assumed for a certain duration of time while reading concentrated from a computer screen, for example, in a comfortable chair.

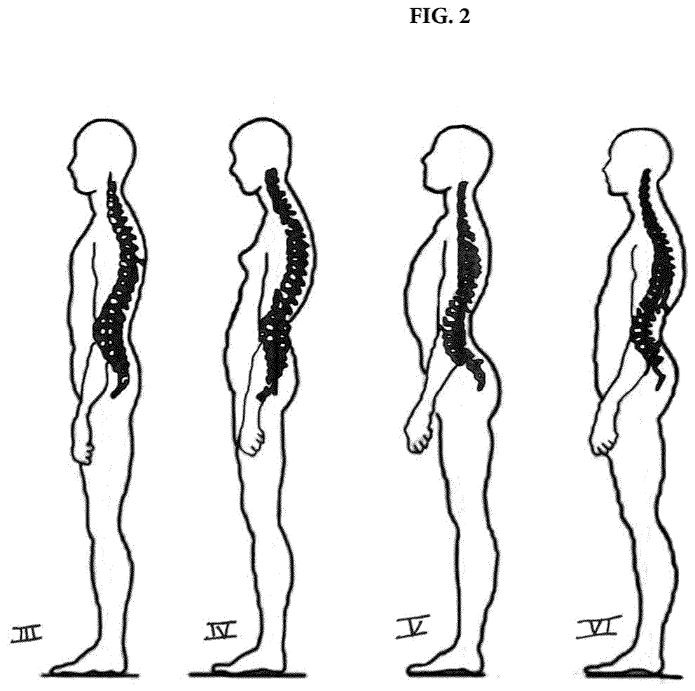

FIG. 2 illustrates various standing postures illustrating the curving of the spine of a user. Posture III illustrates a user having a correct posture standing straight up. In posture IV, the person is slightly slouching and the upper part of the spine is bent too much. In poster V, the lower part of the spine is too much curved putting the vertebrae and muscles under too much stress.

The position in FIG. 2, VI is a combination of the positions IV and V wherein both the person is slouching and has curved his lower back too much. By accurately sensing the position of the shoulders using the sensor devices of the system in accordance with the present invention, additionally each of these incorrect positions IV, V and VI may be detected by the system of the present invention.

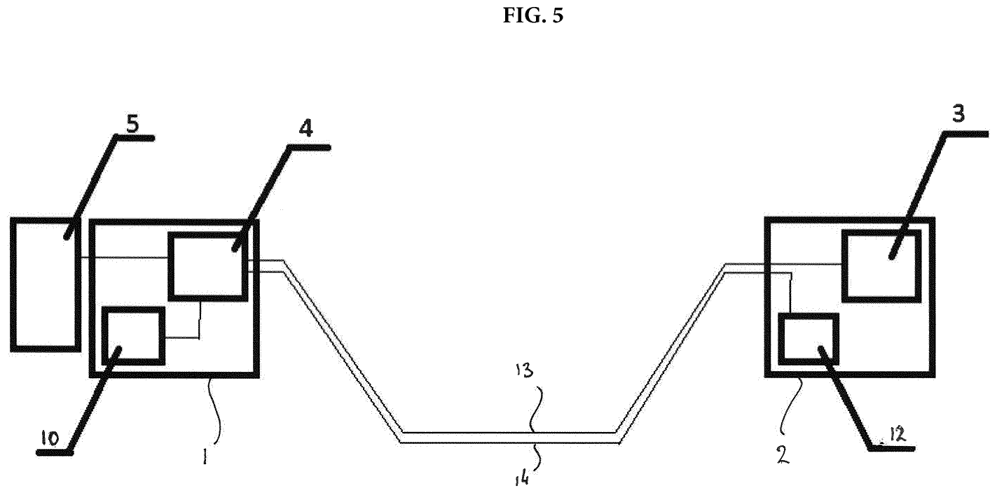

Referring to FIG. 5, the system comprises a first sensor 1 and a second sensor 2, each comprising a printed circuit board (PCB). A first sensor 1 comprises a PCB including a microprocessor 4 serving as controller of the system, a first sensor unit 10, a Bluetooth module 5 and a universal serial bus (USB) port (not shown) for charging the one or more batteries 3. Instead of a USB type charging port, the system may alternatively be equipped with a wireless charging unit. The second sensor 2 comprises a PCB including one or more batteries 3, an on/off switch (not shown) and a second sensor unit 12. Signals from the second sensor unit 12 are conveyed to the microprocessor 4 via data connection 14. Power from the battery 3 is supplied to the first sensor 1 via power line 13. In accordance with some alternative embodiments, each PCB may connected to a battery and a vibrator motor (not shown). The vibrational motor could be installed instead of Bluetooth module 5, in which case such a motor may be implemented on each of the first and second sensor 1 and 2. Yet in other embodiments, a speaker or sound generating device (not shown) may be present in the system to generate an audible signal. The PCB's of the two sensor devices may be connected to each other by means of a wire which includes the power line 13 and data connection 14 such as to exchange signals between the various components. Alternatively, wireless communication may be established between the two PCB's in a manner known to the skilled person, in which case each sensor device 1 and 2 requires it's own power supply 3. Furthermore, although the embodiments show the system, in particular the alarm generator thereof, to comprise a communication device in the form of a Bluetooth module 5, it may be appreciated that any other suitable type of communication device may be applied. For example, the system may alternatively be equipped with a WIFI module to enable data communication with an external device.

The sensor devices 1 and 2 may include any suitable type of sensor that may be applied to locally detect position and orientation data. For example, the sensor devices 1 and 2 may include one or more of the following types of sensors: one or more accelerometers; one or more gyroscopes; one or more gravity sensors; or one or more motion sensors. For example, gravity sensors or gyroscopes may be used to detect specific angling of the sensor devices 1 and 2. Accelerometers and motion sensors may detect motion and motion change.

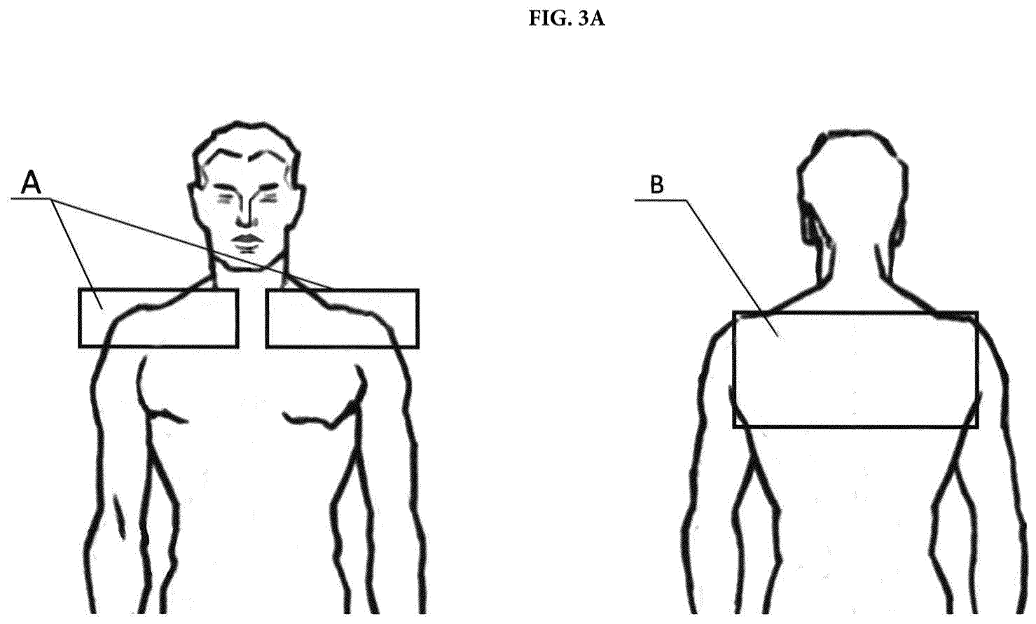

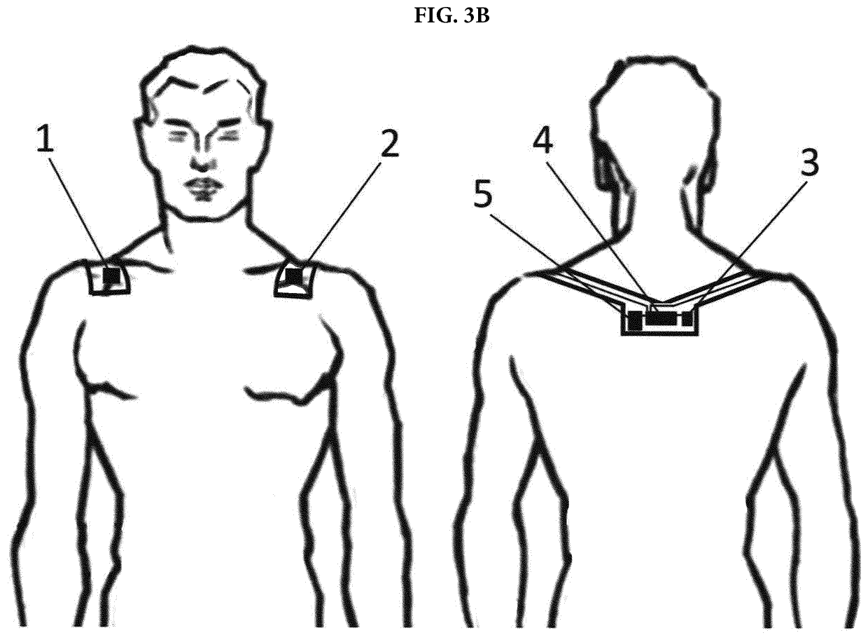

The device is placed on top of a user's shoulders or in an area near the shoulders, i.e. an upper thoracic part of the body, as illustrated in FIG. 3A by regions A and B. The two sensor devices, each including a PCB, battery and vibrating motor are placed such that a sensor device is placed on either side of the user's spine. The optional wire that connects the two sensor devices may be located at the upper back region B in FIG. 3A. An alternative embodiment of the system is schematically illustrated in FIG. 3B, wherein the microprocessor 4, the battery 3 and the Bluetooth module 5 are located in a central part of the system whereas the sensor devices 1 and 2 are remote therefrom.

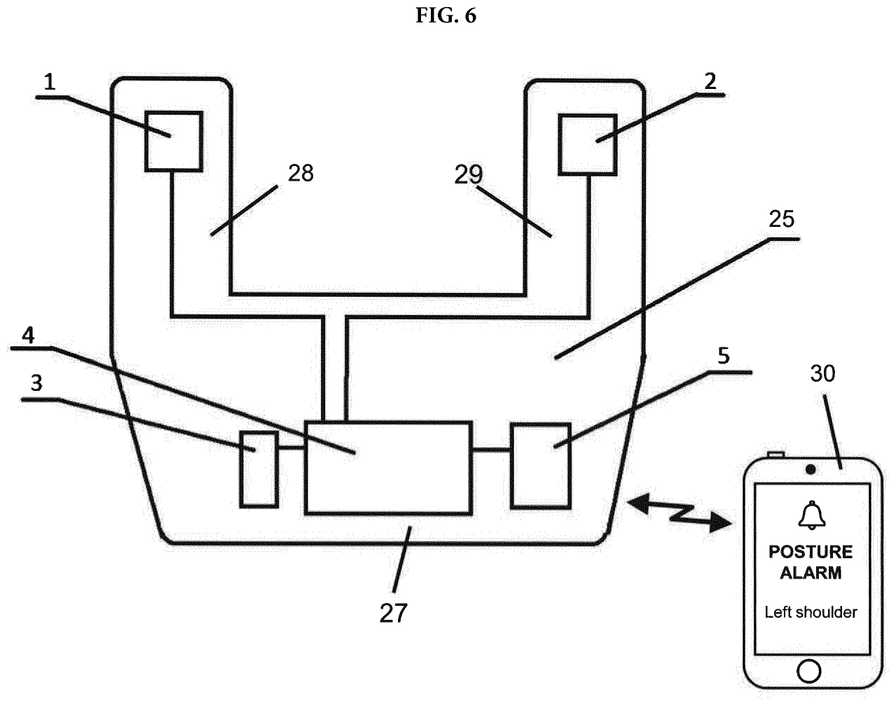

Optionally, the two sets are placed on a semi-rigid surface that follows the shape of the body and shoulders. An embodiment of such a system is illustrated in FIG. 6, wherein the components 1-5 are all mounted on the semi-rigid structure 25. A mid section 27 of the semi-rigid structure 25, in this embodiment, comprises the battery 3, the controller or microprocessor 4, and the Bluetooth module 5. The semi-rigid structure 25 may comprise a first extension 28 and a second extension 29. The first sensor 1 is mounted on the first extension 28 and the second sensor 2 is mounted on the second extension 29. In the embodiment of the system of FIG. 6, the Bluetooth module 5 communicates with a further processing device 30, in particular a mobile phone. Alarm signals generated upon detection of an incorrect shoulder alignment, are transmitted to the mobile phone 30. Software running on the phone 30 processes the received alarm signal and generates an on screen alarm, as illustrated in FIG. 6. Together with the indication on screen, a sound and/or vibration generated by the phone 30 may draw the user's attention to the alarm. The system illustrated in FIG. 6, together with the further processing device 30, provides an exemplary embodiment of an assembly in accordance with the invention.

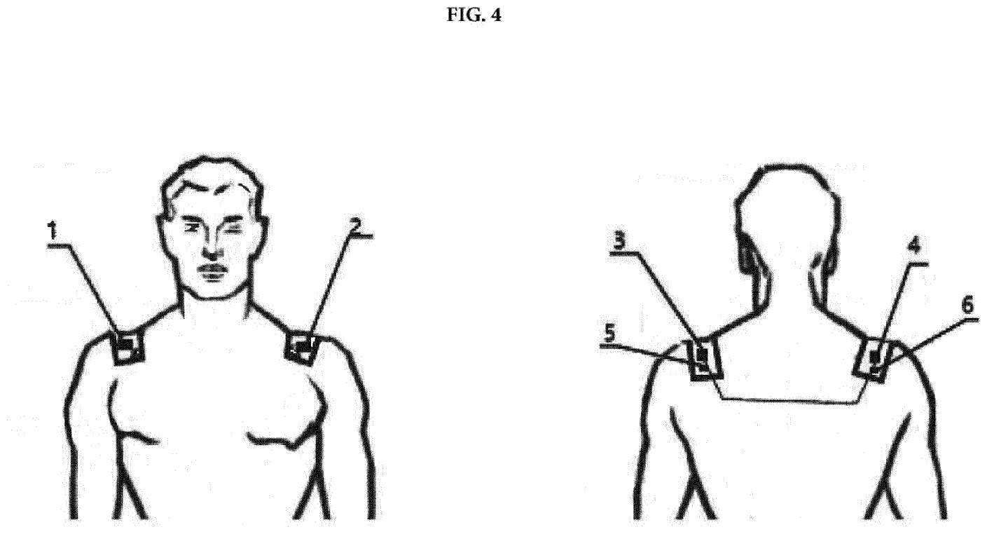

Alternatively, as illustrated in FIG. 4, in some embodiments the semi-rigid structures may be absent and the sensors 1 and 2 are attached directly to the skin, for example in the shoulder area, in which case the sensor devices may be smaller. For the connection between the two devices, this will make no difference. The embodiment illustrated in FIG. 4 shows that each sensor unit 1 and 2 includes a vibrational actuator or motor 6 for providing alarm signals to the user. Here, the controller 4 is further arranged for identifying, based on the first and the second sensor signals, at least one of the shoulders of the user to be deviating from a preferred position associated with shoulder alignment. The controller 4 may then operate an internal alarm generator for generating an alarm signal which is indicative of the shoulder that deviates from the preferred position. This will enable to generate a buzz signal using the vibrational actuator 6 of either the first sensor 1 or the second sensor 2.

The system elements itself may be covered with a soft material, such as silicone, a textile, a gel, a plastic, a foam, or the like. Preferably, though not essential, an adhesive layer is located underneath the sensor devices 1 and 2 or on the semi-rigid structure to facilitate proper attachment. However, alternatively, the sensors 1 and 2 may be located on a stretchable or elastic garment or cloth, such as a band, a braces, or a bra. The sensor devices 1 and 2 may also be mounted on a semi-rigid element configured for replicating the shape of at least a part of the body of the user (e.g. as in FIG. 6). Any of these measures allows the sensors to be maintained in close contact on the skin of the user, increasing accuracy of the measurements.

If the system comprises an alarm generator including a Bluetooth module 5, a connection with the further processing device needs to be established. Hereinafter, in the present example, the further processing device is considered to be a mobile phone. Hence, when the system is correctly placed on or attached to the body, the user should turn on Bluetooth on the mobile phone. The mobile phone has installed thereon suitable software that enables to receive the alarm signals from the alarm generator of the system. Before using device the user has to connect to the device via Bluetooth, and perform a calibration of the system. In some embodiments, the user has to calibrate the system each time it is used. In some more sophisticated embodiments, the controller of the system applies algorithms with mathematical calculation and memorization of previous usages of the system (Machine Learning or Artificial Intelligence), which would make the calibration not necessary for each use.

The calibration process may be performed in the software on the phone, or by the on-board microprocessor 4 of the system. The calibration process, in a first embodiment thereof, consists of two stages. In a first stage the user assumes a correct posture for a period of time. In the second stage this is followed by an incorrect posture of the user. The information (e.g. signals indicative of angles derived from the sensor devices 1, 2) is stored in the memory unit (not shown) of the system. If the system is not equipped with a memory unit, these values may alternatively be stored on the phone or in the cloud, i.e. a storage facility accessed via a communications network. Using a memory of the system, this information may be lost every time the microprocessor is switched off. Alternatively, using a flash memory on the system may allow the information to be available after switching of.

Using the data that was collected during the calibration, the range of angles (data)—allowed range—in which the posture is considered correct is calculated. After calibration, if the user slouches and/or has his shoulders not in alignment, i.e. current data from the sensors 1 and 2 deviates from the allowed range that was calculated during calibration, the controller/microprocessor 4 detects an alignment status of the shoulder that is indicative of misalignment. In that event, the controller 4 triggers the vibrating motor(s) 6 to buzz, or alternatively triggers the Bluetooth module 5 to send a signal to the phone of the user. The preference of being notified through the buzz from the device or/and a notification on the phone is chosen in the software by the user.

An alternative embodiment of the calibration process only consists of the first stage, wherein a user assumes a correct posture for a period of time. The system will record the signal obtained from the first and second sensors 1 and 2. After calibration, the microprocessor 4 of the system may use algorithms that recognize an incorrect posture e.g. by detecting an angle difference with respect to the recorded reference angle, which exceeds a threshold. Not just an angle, but any other of derivable parameters that may be obtained using the signals from sensors 1 and 2, may be matched against one or more thresholds. Even other algorithms may analyze a difference between the signals received from the first and second sensor 1 and 2, such as to detect a misalignment.

Due to the fact that the system has two sensors 1 and 2, it is possible to use the combination of data from two locations (one on each shoulder) in the algorithm. This enables to see the deviation from the allowed range not only when the shoulders are not in alignment, but also when the user slouches. Additionally, the device may also trigger a notification when the user has had his posture correct (i.e. the data from the sensors has been within the allowed range) for a long period of time.

Having sensors 1 and 2 in close contact with skin allows the system to accurately collect the data at any given point of time regardless of the conditions of clothes, activity level of the user. Calculating data from the locations on the shoulder area (both left and right sides of the spine), provides sufficient accuracy in the data to see the deviations in the lower or/and upper back, as well as the shoulders (meaning posture and shoulders).

The present invention has been described in terms of some specific embodiments thereof. It will be appreciated that the embodiments shown in the drawings and described herein are intended for illustrated purposes only and are not by any manner or means intended to be restrictive on the invention. It is believed that the operation and construction of the present invention will be apparent from the foregoing description and drawings appended thereto. It will be clear to the skilled person that the invention is not limited to any embodiment herein described and that modifications are possible which should be considered within the scope of the appended claims. Also kinematic inversions are considered inherently disclosed and to be within the scope of the invention. Moreover, any of the components and elements of the various embodiments disclosed may be combined or may be incorporated in other embodiments where considered necessary, desired or preferred, without departing from the scope of the invention as defined in the claims.

In the claims, any reference signs shall not be construed as limiting the claim. The term ‘comprising’ and ‘including’ when used in this description or the appended claims should not be construed in an exclusive or exhaustive sense but rather in an inclusive sense. Thus the expression ‘comprising’ as used herein does not exclude the presence of other elements or steps in addition to those listed in any claim. Furthermore, the words ‘a’ and ‘an’ shall not be construed as limited to ‘only one’, but instead are used to mean ‘at least one’, and do not exclude a plurality. Features that are not specifically or explicitly described or claimed may be additionally included in the structure of the invention within its scope. Expressions such as: “means for . . . ” should be read as: “component configured for . . . ” or “member constructed to . . . ” and should be construed to include equivalents for the structures disclosed. The use of expressions like: “critical”, “preferred”, “especially preferred” etc. is not intended to limit the invention. Additions, deletions, and modifications within the purview of the skilled person may generally be made without departing from the spirit and scope of the invention, as is determined by the claims. The invention may be practiced otherwise then as specifically described herein, and is only limited by the appended claims.

1. A system for correcting shoulder alignment, the system comprising at least two sensor devices configured to be placed on a body of a user, a controller communicatively connected to the sensor devices such as to receive respective sensor signals therefrom, and an alarm generator configured to generate alarm signals to inform the user of a detected misalignment,
wherein the at least two sensor devices comprise a first sensor to be placed on a first upper thoracic part of the body of the user and a second sensor to be placed on a second upper thoracic part of the body of the user, wherein the first upper thoracic part and the second upper thoracic part are respectively located on different lateral sides of a spine of the user, and wherein the first sensor provides a first sensor signal and the second sensor provides a second sensor signal;
wherein the controller is configured for calculating from at least the first sensor signal and the second sensor signal, an alignment status of the shoulders of the user, and
wherein the controller is further configured for operating the alarm generator for generating an alarm signal when the controller identifies the alignment status to be indicative of a misalignment of the shoulders of the user;
the system further comprising a memory for storing data, wherein the controller is further configured for, in response to receiving a calibration trigger performing a calibration process:
obtaining the first sensor signal and obtaining the second sensor signal;
calculating a reference alignment status from the first and the second sensor signal; and
storing the reference alignment status in the memory.

2. The system according to claim 1, wherein the calibration process includes a first stage wherein:
the step of obtaining the first sensor signal and obtaining the second sensor signal are performed while the user assumes a correct posture for a period of time;
further including recording the first sensor signal and the second sensor signal obtained during the second stage in the memory.

3. The system according to claim 2, wherein the calibration process further includes a second stage wherein:
the step of obtaining the first sensor signal and obtaining the second sensor signal are performed while the user assumes an incorrect posture for a period of time;
further including recording the first sensor signal and the second sensor signal obtained during the second stage in the memory.

4. The system according to claim 1, wherein the first sensor signal and the second sensor signal are indicative of an angle of deviation of alignment of the shoulders of the user.

5. The system according to claim 4, wherein the calibration process further includes calculating, from the obtained first and second sensor signals, a range of angles as an allowed range in which a posture of the user is considered correct.

6. The system according to claim 1, wherein for identifying the alignment status to be indicative of the misalignment, the controller is configured for comparing an actual alignment status with the reference alignment status.

7. The system according to claim 1, wherein the controller is further arranged for identifying, based on the first and the second sensor signal, at least one of the shoulders of the user to be deviating from a preferred position associated with shoulder alignment, wherein the controller is configured for operating the alarm generator for generating the alarm signal such that the alarm signal is indicative of the at least one shoulder that deviates from the preferred position.

8. The system according to claim 7, wherein the alarm generator includes at least a first feedback device and a second feedback device, wherein the first feedback device is associated with the first sensor device, and wherein the second feedback device is associated with the second sensor device, wherein the controller is configured for at least one of:
operating the first feedback device for generating an alarm signal indicative of the first shoulder to be deviating from the preferred position; and
operating the second feedback device for generating an alarm signal indicative of the second shoulder to be deviating from the preferred position.

9. The system according to claim 1, further comprising a memory for storing data, wherein the controller is further configured for:
calculating from at least the first sensor signal and the second sensor signal further posture data indicative of a posture of the user, such as orientation and alignment data of at least a part of a spine of the user; and
comparing the further posture data with reference data from the memory for detecting a deviation from a reference posture.

10. The system according to claim 9, wherein the controller is further configured for operating the alarm generator for generating an alarm signal when the controller identifies that the deviation from the reference posture exceeds a threshold.

11. An assembly of a system according to claim 1 and a further processing device, such as a mobile terminal or handheld device, wherein the alarm generator of the system includes a communication device for transmitting the alarm signal to the further processing device, and wherein the further processing device is configured for:
receiving an alarm signal transmitted by the communication device included in the alarm generator; and
signaling the user, via an output device included on the further processing device, of a detected misalignment of the shoulders.

12. The assembly according to claim 11, wherein the controller of the system is further arranged for identifying, based on the first and the second sensor signal, at least one of the shoulders of the user to be deviating from a preferred position associated with shoulder alignment, and wherein the controller of the system is configured for operating the alarm generator for generating the alarm signal such that the alarm signal is indicative of the at least one shoulder that deviates from the preferred position;
the further processing device being further configured for:
analyzing the alarm signal and provide, via the output device, instructions to the user such as to correct a positioning of the at least one shoulder or a posture of the user.

13. The assembly according to claim 11, wherein the system further comprises a memory for storing data, wherein the controller of the system is further configured for:
calculating from at least the first sensor signal and the second sensor signal further posture data indicative of a posture of the user, such as orientation and alignment data of at least a part of a spine of the user;
comparing the further posture data with reference data from the memory for detecting a deviation from a reference posture; and
operating the alarm generator for generating an alarm signal when the controller identifies that the deviation from the reference posture exceeds a threshold, the alarm signal being indicative of the deviation of the reference posture;
the further processing device being further configured for:
analyzing the alarm signal and provide, via the output device, instructions to the user for correcting the user's posture to decrease the deviation.

14. A computer program product for use in a further processing device, the computer program product residing on a non-transitory computer readable medium, such as a network attached server or data store, the computer program product comprising instructions for causing the further processing device to:
receiving an alarm signal transmitted by a communication device included in an alarm generator of a system for correcting shoulder alignment; and
signaling the user, via an output device included on the further processing device, of a detected misalignment of the shoulders.
